(19) United States
(12) Patent Application Publication
(10) Pub. No.: US 2026/0109735 A1
(43) Pub. Date: Apr. 23, 2026

(54) OCTAPEPTIDE, PREPARATION METHOD THEREOF, AND APPLICATION THEREOF IN ANTI-BACTERIUM AND IMMUNOMODULATION

(71) Applicants: Chongqing Academy Of Animal Sciences, Chongqing (CN); Technology Innovation Center for Pigs (Chongqing), Chongqing (CN)

(72) Inventors: Feiyun Yang, Chongqing (CN); Jinxiu Huang, Chongqing (CN); Xiuliang Ding, Chongqing (CN)

(21) Appl. No.: 19/317,121

(22) Filed: Sep. 2, 2025

(30) Foreign Application Priority Data
Sep. 20, 2024 (CN) 202411311150.4

(51) Int. Cl. C07K 7/64; A61K 38/00; A61P 31/04; C12P 21/02; G01N 33/68

(52) U.S. Cl. CPC C07K 7/64; A61P 31/04; C12P 21/02; G01N 33/6851; A61K 38/00

(57) ABSTRACT

The disclosure provides an immunomodulatory octapeptide, and this small peptide molecule has a unique structure, higher broad-spectrum antibacterial activity, stronger pathogenic bacteria inhibition and high purity, can maintain stability of its structure and antibacterial activity in artificial gastric juice, with no obvious hemolytic activity, and has high application value in animal husbandry. The disclosure provides a preparation method and a purity test method of this immunomodulatory octapeptide.

### CROSS-REFERENCE TO RELATED APPLICATIONS

This application claims priority to Chinese Patent Application No. 202411311150.4, filed on Sep. 20, 2024, which is hereby incorporated by reference in its entirety.

### SEQUENCE LISTING

The present application contains a sequence listing which was filed electronically in XML format and is hereby incorporated by reference in its entirety. Besides, the XML copy is created on Dec. 26, 2025, is named “OCTAPEPTIDE, PREPARATION METHOD THEREOF, AND APPLICATION THEREOF-Sequence Listing” and is 6,045 bytes in sizes.

### TECHNICAL FIELD

The disclosure relates to the field of biotechnology, in particular to an octapeptide, a preparation method thereof, and application thereof.

### BACKGROUND

Lipopeptides or polypeptides are very important compounds in microbial secondary metabolites. They have attracted much attention from researchers because of their unique chemical structures and diverse biological activities. The lipopeptides or polypeptides are polymerization products of amino acids which lie between amino acids and protein, and are substantially synthesized by ribosomes or multi-module non-ribosomal peptide synthetases. Immunomodulatory octapeptide can be generated by expressing brevibacterium laterosporus and is a secondary metabolite of non-ribosomal peptides of an octapeptin family. Bacillus laterosporus is a type of gram-negative bacilli, with elongate and rod-shaped thallus, strong adaptability and high-temperature resistance. Meanwhile, this strain can produce a variety of bioactive substances including lipopeptides and polypeptides.

Appearance of traditional antibiotics not only makes the pharmaceutical industry develop rapidly, but also has very important application in livestock and poultry breeding. However, due to abuse of the traditional antibiotics and emergence of multi-drug resistant bacteria, development of the animal husbandry is seriously affected and ecological environment and human health are endangered. In order to solve this problem, it is urgent to develop new antibiotic substitute products for animal husbandry.

### SUMMARY

In view of above problems, an immunomodulatory octapeptide with a unique structure, excellent activity, strong specificity, high purity, high in-vivo adaptability, stability and safety is provided in the disclosure.

In order to achieve the above object, technical schemes adopted by the disclosure are as follows.

An octapeptide is also provided in the disclosure, with a structural formula as follows:

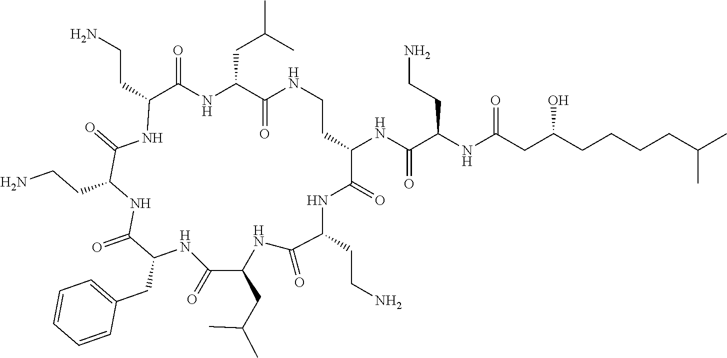 [SMILES: CC(C)CCCC[C@@H](O)CC(=O)N[C@H](CCN)C(=O)N[C@H]1CCNC(=O)[C@@H](CC(C)C)NC(=O)[C@@H](CCN)NC(=O)[C@@H](CCN)NC(=O)[C@@H](Cc2ccccc2)NC(=O)[C@H](CC(C)C)NC(=O)[C@@H](CCN)NC1=O]

The octapeptide is prepared by fermentation of brevibacterium laterosporus. The brevibacterium laterosporus is preferably Bacillus laterosporus M811 developed by the applicant, which is preserved in China Center for Type Culture Collection, with a preservation date of Apr. 25, 2024, an address of Wuhan University in Wuhan, China, and a preservation number of CCTCC NO: M2024715. This strain is used to prepare the immunomodulatory octapeptide, which improves yield, purity, activity and adaptability of products.

A preparation method of the octapeptide is provided, which includes:
(1) fermenting brevibacterium laterosporus to prepare fermentation liquid;
(2) preparing a crude fermentation extract by salt ion precipitation; and
(3) separating and purifying the crude fermentation extract by high-performance liquid chromatography, removing acetonitrile by rotary evaporation, and freeze-drying.

Preferably, a preparation process of the high-performance liquid chromatography in step (3) is performed with a preparative liquid chromatographic column, Agilent Zorbax 300SB-C18 PrepHT; a flow of water containing 0.1% of trifluoroacetic acid (A) and acetonitrile containing 0.1% of trifluoroacetic acid (B); and
an elution mode being shown in Table 1:

TABLE 1
Elution gradient for octapeptide analysis

| Time | | |
|---|---|---|
| (min) | A, % | B, % |
| 0 | 70 | 30 |
| 57 | 63.4 | 36.6 |
| 58 | 0 | 100 |
| 64 | 0 | 100 |
| 65 | 70 | 30 |
| 67 | 70 | 30 |

with a flow rate of 5 mL/min, column temperature of 30° C., and a detection wavelength of 220 nm.

Preferably, the step (2) specifically includes: adjusting pH of the fermentation liquid obtained in the step (1) to 3.0 with 5 M H2SO4, centrifuging at 8000 rpm for 5 min, and reserving supernatant; and adding ammonium sulfate with a concentration of 5 to 30 g/mL to the supernatant, slowly stirring for 30 min, then centrifuging at 8000 rpm for 10 min, and collecting precipitate for drying at 60° C. to obtain the crude fermentation extract. The immunomodulatory octapeptide can be precipitated and enriched by ammonium sulfate of a specific concentration, and different types of proteins can be precipitated by ammonium sulfate with different concentrations. In the disclosure, an optimal concentration of ammonium sulfate is added to the fermentation liquid obtained in the step (1), so that the immunomodulatory octapeptide can be precipitated and enriched to a greatest extent to obtain an optimal recovery rate, and meanwhile, interference of foreign proteins on subsequent preparative liquid chromatography separation and purification steps can be removed, and purity of pure immunomodulatory octapeptide can be improved.

A method for preparing immunomodulatory octapeptide by using brevibacterium laterosporus specifically includes:
(1) inoculating bacillus laterosporus M811 in NB slant culture medium for activation and culturing at 37° C. for 24 h, and then taking a single colony to inoculate in a sterilized NB liquid culture medium shake flask for culturing at 37° C. and 220 rpm for 24 h to obtain a primary seed solution, fully mixing and transferring the primary seed solution to culture medium for a secondary seed solution with an inoculation amount of 5% for culturing at 37° C. and 220 rpm for 24 h to obtain the secondary seed solution; and inoculating the secondary seed solution into optimized fermentation medium with an inoculation amount of 5% by volume for culturing at 30° C. and 220 rpm for 48 h to obtain the fermentation liquid;
(2) adjusting a pH value of the resulting fermentation liquid obtained in step (1) to 3.0, centrifuging at 8000 rpm for 5 min, and reserving supernatant; and adding ammonium sulfate powder of 5 to 30 g/mL into the supernatant, slowly stirring for 30 min, centrifuging to collect precipitate and baking at 60° C. to obtain a crude extract of the immunomodulatory octapeptide; and
(3) dissolving the crude extract obtained in step (2) with an initial flow of preparative high-performance liquid phase to a concentration of 0.1 g/mL, centrifuging to obtain supernatant, and separating and purifying the supernatant with the preparative high-performance liquid chromatograph; and collecting a fraction corresponding to the immunomodulatory octapeptide, removing acetonitrile by rotary evaporation, and freeze-drying to obtain the pure octapeptide product. Purity test is performed by analytical high-performance liquid chromatography.

Preferably, the culture medium for the secondary seed solution described in step (1) is with 3 g/L of peptone, 16 g/L of yeast powder, 2 g/L of sucrose, 0.1 g/L of magnesium sulfate, and pH of 6.8±0.1. The fermentation medium is with 15 g/L of corn syrup powder, 10 g/L of sucrose, 3 g/L of ammonium sulfate, 0.3 g/L of magnesium sulfate, and pH of 6.8±0.1.

A method for detecting purity of the octapeptide is further provided, which includes:
placing 1 mg of the pure octapeptide product obtained in the step (3) in a centrifuge tube and dissolving it with 1 mL of ultrapure water, vortexing for 1 min and then passing through a 0.22 μm filter membrane, and detecting the purity of the immunomodulatory octapeptide by the analytical high-performance liquid chromatography. Preferably, an analysis process of the analytical high-performance liquid chromatography is performed with an analytical liquid chromatography column, Sepax Bio-C18 4.6×250 mm 5 μm; and a flow of water containing 0.1% of trifluoroacetic acid (A) and acetonitrile containing 0.1% of trifluoroacetic acid (B);
an elution mode being shown in Table 2:

TABLE 2
Elution gradient for octapeptide analysis

| Time | | |
|---|---|---|
| (min) | A, % | B, % |
| 0 | 80 | 20 |
| 4 | 80 | 20 |
| 10 | 50 | 50 |
| 13 | 40 | 60 |
| 15 | 0 | 100 |
| 19 | 0 | 100 |
| 22 | 80 | 20 |
| 26 | 80 | 20 |

with a flow rate of 1 mL/min, column temperature of 30° C., and a detection wavelength of 220 nm.

The disclosure also provides application of the octapeptide in antibiotics or immunomodulators, or in preparation of medicines, reagents, feeds and feed additives. Preferably, the octapeptide is an anti-Enterobacter octapeptide, and its application in preparing drugs or feed additives against gram-negative enterobacter infection. The gram-negative Enterobacter includes but is not limited to Escherichia coli, Salmonella, Shigella dysenteriae and Pseudomonas aeruginosa. With screening of antibacterial activity, the octapeptide has good activity of inhibiting pathogenic enterobacteria such as Escherichia coli, Salmonella, Shigella dysenteriae and Pseudomonas aeruginosa, and can be used for development and utilization of antibacterial drugs, leading veterinary drugs and feed additives for inhibiting related diseases and infections caused by various Gram-negative pathogenic enterobacteria.

### Beneficial Effects

1. The disclosure provides an octapeptide with gram-negative and gram-positive pathogenic bacteria resistance, and this small peptide molecule has a unique structure, higher broad-spectrum antibacterial activity, stronger pathogenic bacteria inhibition, high purity and strong performance stability.
2. The immunomodulatory octapeptide provided in the disclosure can maintain stability of its structure and antibacterial activity in artificial gastric juice, with no obvious hemolytic activity. The immunomodulatory octapeptide has strong bactericidal effect on both gram-positive and gram-negative pathogenic bacteria, and its bactericidal ability on Escherichia coli and Pseudomonas aeruginosa is obviously stronger than that on Staphylococcus aureus, and thus it has high application potential in animal husbandry.
3. The disclosure provides the preparation method of the immunomodulatory octapeptide, in which a high-purity finished octapeptide product is prepared through multi-stage seed liquid culturing, fermentation liquid preparation, fermentation liquid preprocessing, octapeptide extraction and refining, which better improves fermentation efficiency and shortens a separation and purification process.
4. The disclosure uses combination of salt ion precipitation and preparative reversed-phase high-performance liquid chromatography to treat the octapeptide fermentation liquid, which improves preparation efficiency and sample purity of the octapeptide and reduces preparation cost. Purity of the octapeptide prepared by this method is as high as 98.23%.

### BRIEF DESCRIPTION OF DRAWINGS

FIG. 1 is a phylogenetic tree for identification of Bacillus laterosporus M811 strain in Example 1 of the disclosure;

FIG. 2 shows a detection result of a recovery rate of octapeptide in Example 2 of the disclosure;

FIG. 3 is a HPLC preparation diagram of octapeptide in Example 2 of the disclosure;

FIG. 4 is a purity test diagram of octapeptide in Example 2 of the disclosure;

FIG. 5 is a HPLC preparation diagram of octapeptide in Example 3 of the disclosure;

FIG. 6 is a purity test diagram of octapeptide in Example 3 of the disclosure;

FIG. 7 is a HPLC preparation diagram of octapeptide in Example 4 of the disclosure;

FIG. 8 is a purity test diagram of octapeptide in Example 4 of the disclosure;

FIG. 9 is a primary mass spectrum of octapeptide in Example 5 of the disclosure;

FIG. 10 is a secondary mass spectrum of octapeptide in Example 5 of the disclosure;

FIG. 11 is a GC-MS spectrum of fatty acid of octapeptide hydrolysate in Example 5 of the disclosure;

FIG. 12 shows stability of octapeptide in artificial gastric juice in Example 7 of the disclosure; and

FIG. 13 shows hemolytic results of octapeptide in Example 8 of the present disclosure.

### DESCRIPTION OF EMBODIMENTS

Strain used in the disclosure is brevibacterium laterosporus separated from soil in Hewanzi Village, Yongchuan District, Chongqing (N 29°19′54″, E 105°41′11″), which was preserved in China Center for Type Culture Collection on Apr. 25, 2024, with an address of Wuhan University in Wuhan, China and a preservation number of CCTCC NO: M2024715.

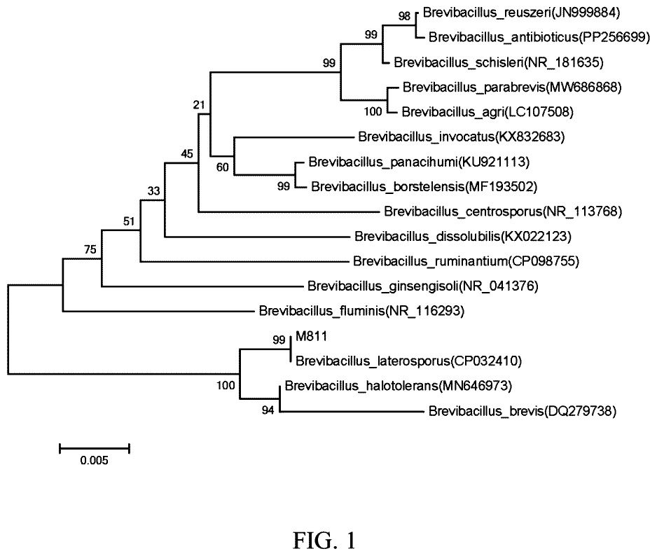

The strain separated in the disclosure is identified to have morphological features that its colony is transparent beige with a shiny surface, its thallus is elongate and rod-shaped and about 2.0×1.0 μm in size, with peritrichous flagella, can form oval spores, and is gram-positive. 16S rRNA gene of this strain is amplified by PCR, sequenced and phylogenetic analyzed, as shown in FIG. 1. It is identified to be brevibacterium laterosporus, named Brevibacillus Laterosporus M811.

NB culture medium used in the disclosure is composed of 10 g/L of tryptone, 5 g/L of yeast extract and 10 g/l of sodium chloride, which are purchased from Beijing Solarbio Science&Technology Co., Ltd. Corn syrup dry powder, sucrose and magnesium sulfate medium components are all commercially available medium components.

Fermentation and compound separation and purification parameters involved in the disclosure are well known to those skilled in the art.

### Example 1

Identification of brevibacterium laterosporus strain for preparing immunomodulatory octapeptide specifically includes following steps.

### (1) DNA Extraction

The DNA extraction was performed using a TSINGKE plant DNA extraction kit (universal), which specifically includes following steps as follows.

1. A Spin Column was placed in Collection Tube, 250 μL Buffer BL was added, and centrifugation was made at 12000 rpm for 1 min to activate a silica gel membrane.

2. Dry tissue (not more than 20 mg) of samples was taken with liquid nitrogen added to grind fully. After grinding, the tissue was placed in a 1.5 mL centrifuge tube, with 400 μL Buffer gP1 being added for vortexing for 1 min, which was placed in a water bath at 65° C. for 10 to 30 min, during which it can be taken out and mixed upside down to fully crack.

3. 150 μL Buffer gP2 was added for vortexing for 1 min and placing in an ice bath for 5 min.

4. Centrifuge was made at 12,000 rpm for 5 min, and supernatant was transferred to a new centrifuge tube.

5. anhydrous ethanol with a same volume as the supernatant was added to immediately shake and mix well, all liquid was transferred into the Spin Column to centrifuge at 12,000 rpm for 30 s, with waste liquid being discarded.

6. 500 μL Buffer Pw was added to the Spin Column (with anhydrous ethanol being added before use) to centrifuge at 12,000 rpm for 30 s, with waste liquid being discarded.

7. 500 μL Wash Buffer was added to the Spin Column (with anhydrous ethanol being added before use) to centrifuge at 12,000 rpm for 30 s, with waste liquid being discarded.

8. Repeat step 7.

9. The Spin Column was placed back into the Collection Tube to centrifuge at 12,000 rpm for 2 min, then its cover was opened for drying for 1 min

10. The Spin Column was taken out and placed into a clean centrifuge tube, with 50 to 100 μl TE Buffer (with the TeBuffer being preheated at 65° C.) being added at a center of an adsorption membrane to leave at 20 to 25° C. for 2 min and centrifuge at 12,000 rpm for 2 min.

### (2) Amplification by PCR

1. Universal primers for bacterial species identification

| | | | Amplified | PCR |
|---|---|---|---|---|
| Category | Name | Sequence 5′→3′ | sequence | length/bp |
| Bacteria | 27F | 27F: | 16S | about |
| | | GAGAGTTTGATCCTGGCTCAG | rDNA | 1500 bp |
| | 1492R | 1492R: | | |
| | | TACGGCTACCTTGTTACGAC | | |

2. Extracted DNA samples were diluted appropriately and used as a PCR template to amplify with 1×TSE101 gold medal mix from TSINGKE with components of an amplification system as follows:

| | |
|---|---|
| 1 × TSE101gold medal | 45 μL |
| mix | |
| 27F (10P) | 2 μL |
| 1492R (10P) | 2 μL |
| DNA template | 1 μL |

Above amplification system amplifies according to following amplification procedures.

| | Temperature | | | Number of |
|---|---|---|---|---|
| Stages | (° C.) | Time | | cycles |
| Predegeneration | 98 | 3 | min | |
| Cycling | 98 | 10 | s | 39 cycles |
| stage | 55 | 15 | s | |
| | 72 | 15 | s/kb | |
| Extension | 72 | 5 | min | |

| | | |
|---|---|---|
| stage | | |
| Preservation | 4 | — |
| stage | | |

### (3) Electrophoretic Measurement

Amplified PCR products were subjected to agarose gel electrophoresis (with 2 μL of samples and 6 μL of bromophenol blue) at a voltage of 300 V for 12 minutes to obtain an identification electropherogram. The prepared PCR products were subjected to first-generation sequencing (with a sequencing primer of 722F/907R).

### (4) Identification

1. ContigExpress was used to splice sequencing results, and inaccurate parts at both ends are removed.

2. Spliced sequences are compared in the NCBI database (blast.ncbi.nlm.nih.gov).

3. In comparison results, a top ranked specie with highest homology of more than 97% and clear specie information obtained by comparison on NCBI was generally used as a reference specie for identification. As shown in FIG. 1, a 16S sequence comparison result of the identified strain sample is Brevibacillus Laterosporus (or belongs to a same genus), and thus it is named Brevibacillus Laterosporus M811.

### Example 2

A preparation method of the immunomodulatory octapeptide specifically includes following steps.

### (1) Fermentation

Bacillus laterosporus M811 was inoculated in NB slant culture medium to culture at 37° C. for 24 h for activation, and a single colony was picked to inoculate in NB liquid culture medium for culturing at 37° C. and 220 rpm for 24 h to obtain a primary seed solution.

The primary seed solution was transferred to culture medium for a secondary seed solution with an inoculation amount of 5% for culturing at 37° C. and 220 rpm for 24 h to obtain the secondary seed solution. The culture medium for the secondary seed solution is with 3 g/L of peptone, 16 g/L of yeast powder, 2 g/L of sucrose, 0.1 g/L of magnesium sulfate, and pH of 6.8±0.1.

The secondary seed solution was inoculated into optimized fermentation medium with an inoculation amount of 5% for culturing at 30° C. and 220 rpm for 48 h to obtain the fermentation liquid. The fermentation medium is with 15 g/L of corn syrup powder, 10 g/L of sucrose, 3 g/L of ammonium sulfate, 0.3 g/L of magnesium sulfate, and pH of 6.8±0.1.

### (2) Preparing Crude Fermentation Extract by Salt Ion Precipitation

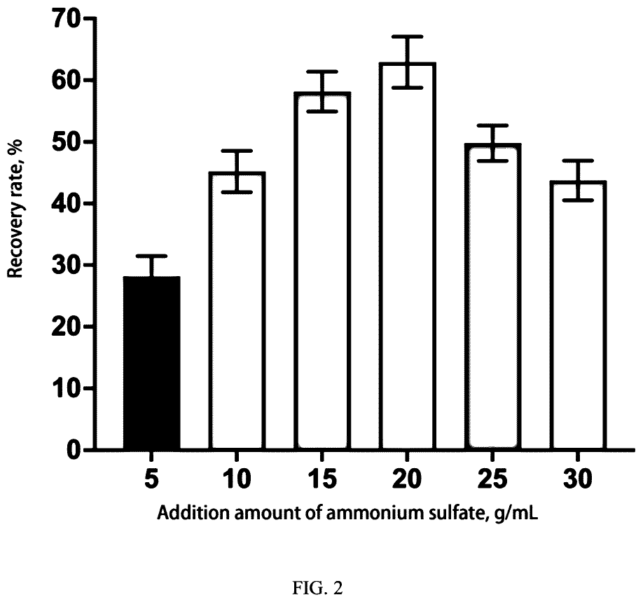

After the fermentation of the immunomodulatory octapeptide, a pH value of the fermentation liquid was adjusted to 3.0 with 0.5 M sulfuric acid, and bacteria were removed by centrifugation at 8000 rpm for 5 min, and supernatant was reserved. Ammonium sulfate powder of 5, 10, 15, 20, 25 and 30 g/mL was slowly added into the supernatant for slowly stirring for 30 min, centrifuging to collect precipitate and baking at 60° C. to obtain a crude extract of the immunomodulatory octapeptide. Recovery rates of the octapeptide precipitated by ammonium sulfate of different concentrations are shown in FIG. 2. It can be seen that when an addditon amount of ammonium sulfate is 20 g/mL, the recovery rate of the immunomodulatory octapeptide reaches the maximum, which is 62.93±5.87%. In optimization of subsequent experimental conditions, 20 g/mL of ammonium sulfate was used to prepare the crude extract of the immunomodulatory octapeptide.

### Calculation Formula of Recovery Rate:

$$Recovery rate = \frac{Amount of immunomodulatory octapeptide in crude extract}{Amount of immunomodulatory octapeptide in supernatant} \times 100 \%$$

### (3) Separation and Purification

The crude extract was diluted to 0.1 g/mL by an initial flow of preparative liquid phase for centrifuging. Obtained supernatant was separated and purified by preparative high-performance liquid chromatography (Agilent, 1260 Infinity II), with a preparation process performed with: a preparative liquid chromatography column, Agilent Zorbax 300SB-C18 PrepHT; a flow of water containing 0.1% of trifluoroacetic acid (A) and acetonitrile containing 0.1% of trifluoroacetic acid (B); and
an elution mode being shown in Table 3:

TABLE 3
Elution gradient for octapeptide preparation

| Time | | |
|---|---|---|
| (min) | A, % | B, % |
| 0 | 80 | 20 |
| 4 | 72 | 28 |
| 8 | 66 | 34 |
| 23 | 66 | 34 |
| 35 | 62 | 38 |
| 42 | 59 | 41 |
| 45 | 0 | 100 |
| 52 | 0 | 100 |
| 53 | 80 | 20 |
| 55 | 80 | 20 |

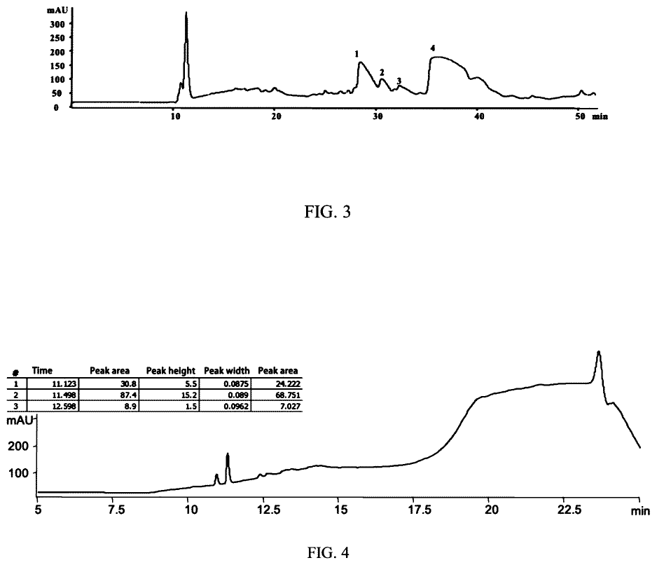

with a flow rate of 5 mL/min, column temperature of 30° C., and a detection wavelength of 220 nm. The preparative chromatography is shown in FIG. 3. A marked peak No. 1 is a chromatographic peak corresponding to the octapeptide.

A fraction corresponding to the octapeptide was collected for rotary evaporation to remove acetonitrile (a RE-3000B rotary evaporator from Shanghai Yarong) and then for freeze-drying (a Millrock box-type freeze-dryer REVOTM) to obtain pure octapeptide products.

### (4) Purity Analysis and Measurement

1 mg of octapeptide was dissolved with 1 mL of ultrapure water to vortex for 1 min and then pass through a 0.22 μm membrane for filtering, and purity analysis was performed by the high-performance liquid chromatography (Agilent, 1260 Infinity II). A chromatography column used in analysis is Sepax Bio-C18 4.6×250 mm 5 μm; and the flow used is water containing 0.1% of trifluoroacetic acid (A) and acetonitrile containing 0.1% of trifluoroacetic acid (B).

An elution mode is shown in Table 4:

TABLE 4
Elution gradient for octapeptide analysis

| Time | | |
|---|---|---|
| (min) | A, % | B, % |
| 0 | 80 | 20 |
| 4 | 80 | 20 |
| 10 | 50 | 50 |
| 13 | 40 | 60 |
| 15 | 0 | 100 |
| 19 | 0 | 100 |
| 22 | 80 | 20 |
| 26 | 80 | 20 |

with a flow rate of 1 mL/min, column temperature of 30° C., and a detection wavelength of 220 nm.

The purity was measured by analytical reversed-phase high-performance liquid chromatography, with results being shown in FIG. 4. Purity of the sample of the immunomodulatory octapeptide prepared in Example 2 was 68.75%.

### Example 3

A preparation method of the immunomodulatory octapeptide specifically includes following steps.

(1) A fermentation step, which is the same as that in Example 1.

(2) After the fermentation of the immunomodulatory octapeptide, a pH value of the fermentation liquid was adjusted to 3.0 with 0.5 M sulfuric acid, and bacteria were removed by centrifugation at 8000 rpm for 5 min, and supernatant was reserved. Ammonium sulfate powder of 20 g/mL was slowly added into the supernatant for slowly stirring for 30 min, centrifuging to collect precipitate and baking at 60° C. to obtain a crude extract of the immunomodulatory octapeptide.

(3) Separation and purification

The crude extract was diluted to 0.1 g/mL by an initial flow of preparative liquid phase for centrifuging. Obtained supernatant was separated and purified by preparative high-performance liquid chromatography (Agilent, 1260 Infinity II), with a preparation process performed with: a preparative liquid chromatography column, Agilent Zorbax 300SB-C18 PrepHT; a flow of water containing 0.1% of trifluoroacetic acid (A) and acetonitrile containing 0.1% of trifluoroacetic acid (B); and
an elution mode being shown in Table 5:

TABLE 5
Elution gradient for octapeptide preparation

| Time | | |
|---|---|---|
| (min) | A, % | B, % |
| 0 | 70 | 30 |
| 57 | 63.4 | 36.6 |
| 58 | 0 | 100 |
| 64 | 0 | 100 |
| 65 | 70 | 30 |
| 67 | 70 | 30 |

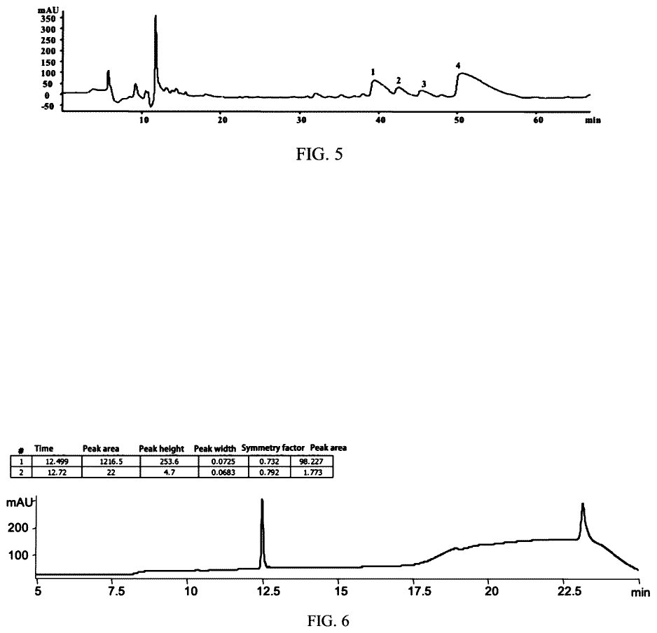

with a flow rate of 5 mL/min, column temperature of 30° C., and a detection wavelength of 220 nm. The preparative chromatography is shown in FIG. 5. A marked peak No. 1 is a chromatographic peak corresponding to the octapeptide.

A fraction corresponding to the octapeptide was collected for rotary evaporation to remove acetonitrile (a RE-3000B rotary evaporator from Shanghai Yarong) and then for freeze-drying (a Millrock box-type freeze-dryer REVOTM) to obtain pure octapeptide products.

(4) A purity detection step, which is the same as that in Example 2. The purity was measured by analytical reversed-phase high-performance liquid chromatography, with results being shown in FIG. 6. Purity of the sample of the immunomodulatory octapeptide prepared in Example 3 was 98.23%.

Experimental results show that the purity of the immunomodulatory octapeptide can be significantly improved by optimizing elution gradient and elution time of the flow in separating and purifying the crude fermentation extract of the disclosure.

### Example 4

A preparation method of the immunomodulatory octapeptide specifically includes following steps.

(1) A fermentation step, which is the same as that in Example 3.

(2) A preparation step of crude extract, which is the same as that in Example 3.

(3) Separation and purification

The crude extract was diluted to 0.1 g/mL by an initial flow of preparative liquid phase for centrifuging. Obtained supernatant was separated and purified by preparative high-performance liquid chromatography (Agilent, 1260 Infinity II), with a preparation process performed with: a preparative liquid chromatography column, Agilent Zorbax 300SB-C18 PrepHT; a flow of water containing 0.1% of trifluoroacetic acid (A) and acetonitrile containing 0.1% of trifluoroacetic acid (B); and an elution mode being shown in Table 6:

TABLE 6
Elution gradient for octapeptide preparation

| Time | | |
|---|---|---|
| (min) | A, % | B, % |
| 0 | 70 | 30 |
| 70 | 62 | 38 |
| 71 | 0 | 100 |
| 77 | 0 | 100 |
| 78 | 70 | 30 |
| 80 | 70 | 30 |

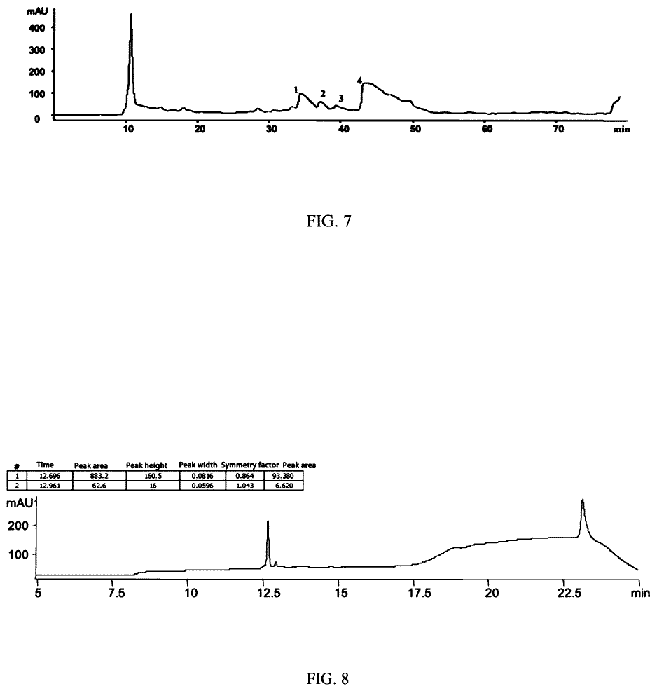

with a flow rate of 5 mL/min, column temperature of 30° C., and a detection wavelength of 220 nm. The preparative chromatography is shown in FIG. 7. A marked peak No. 1 is a chromatographic peak corresponding to the octapeptide.

A fraction corresponding to the octapeptide was collected for rotary evaporation to remove acetonitrile (a RE-3000B rotary evaporator from Shanghai Yarong) and then for freeze-drying (a Millrock box-type freeze-dryer REVOTM) to obtain pure octapeptide products.

(4) A purity detection step, which is the same as that in Example 2. The purity was measured by analytical reversed-phase high-performance liquid chromatography, with results being shown in FIG. 8. Purity of the sample of the immunomodulatory octapeptide prepared in Example 4 was 93.38%. Experimental results show that in separation and purification of the crude fermentation extract of the disclosure, further increasing the elution time of the flow can reduce the purity of the immunomodulatory octapeptide. Therefore, a high-performance liquid phase preparation method constructed in Example 3 is with an optimal separation and purification elution phase, optimal elution gradient and optimal elution time of the immunomodulatory octapeptide.

### Example 5 Molecular Structure Identification of Immunomodulatory Octapeptide

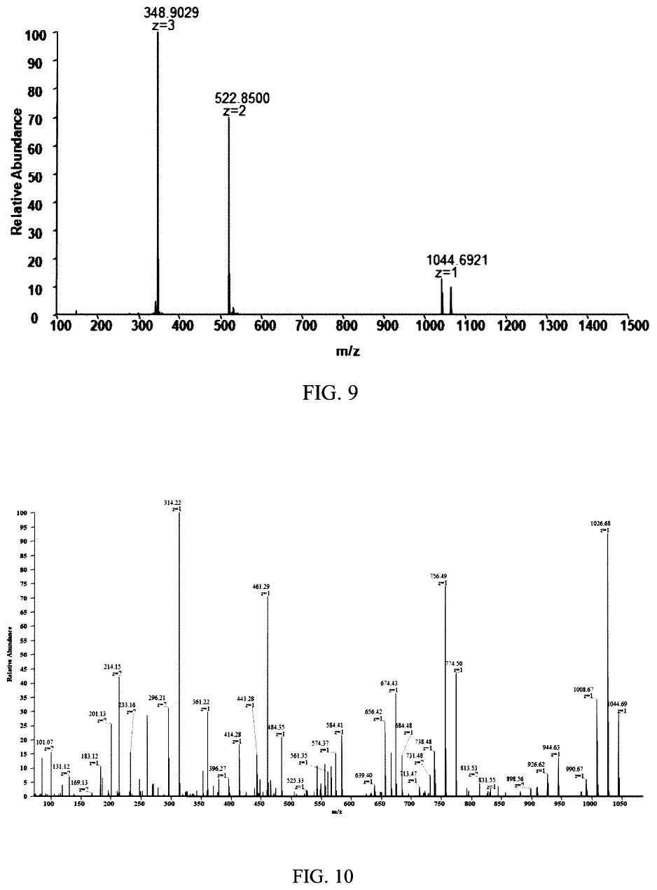

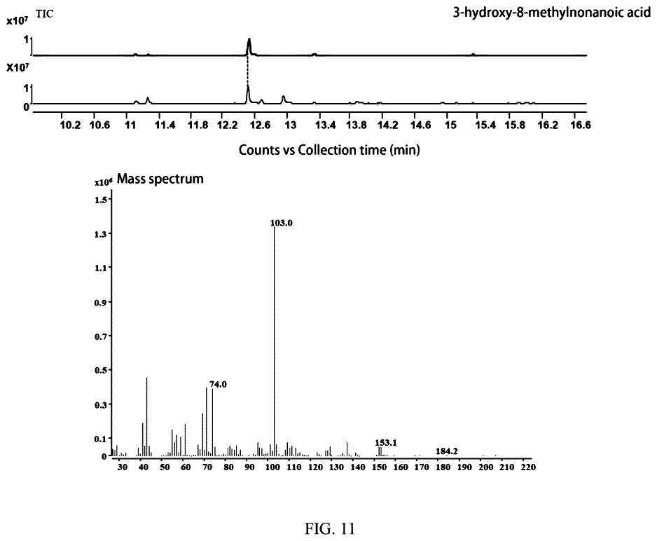

The molecular structure was identified by high resolution mass spectrometry and fatty acid derivatization reaction, with results being shown in FIGS. 9 to 11. High-resolution mass spectrometry in a positive ion mode (HR-ESI-MS) shows ion peaks of octapeptide molecule at m/z 1044.6921 [M+H]+, m/z 522.8500 [M+2H]2+ and m/z 348.9029 [M+3H]3+, and its molecular formula is presumed to be C51H89N13O10 (see FIG. 9). Results of primary and secondary mass spectrometry information and GC-MS analysis of fatty acid derivative products (FIGS. 10 and 11) prove that a prepared sample of the disclosure is octapeptide shown with a following structural formula.

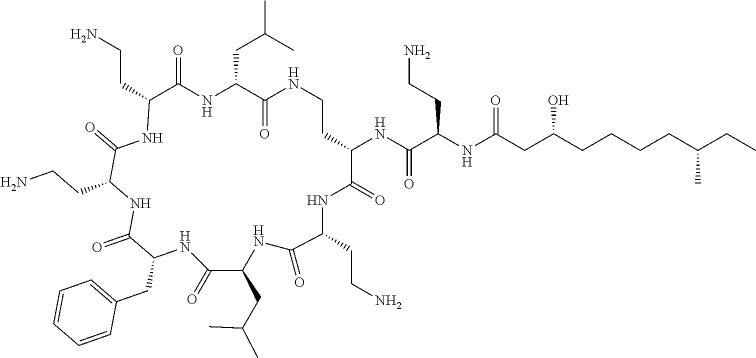 [SMILES: CC[C@H](C)CCCC[C@@H](O)CC(=O)N[C@H](CCN)C(=O)N[C@H]1CCNC(=O)[C@@H](CC(C)C)NC(=O)[C@@H](CCN)NC(=O)[C@@H](CCN)NC(=O)[C@@H](Cc2ccccc2)NC(=O)[C@H](CC(C)C)NC(=O)[C@@H](CCN)NC1=O]

### Example 6 Antibacterial Activity of Immunomodulatory Octapeptide

According to National Committee for Clinical Laboratory Standards, the octapeptide (with a sample source: Example 3) was serially double-diluted to concentrations of 512, 256, 128, 64, 32, 16, 8, 4, 2, 1 and 0.5 μg/mL with MH medium (Mueller Hinton Broth medium from Qingdao Hi-tech Industrial Park Hope Bio-technology Co., Ltd.). 50 μL of diluted octapeptide was added to each well of a 96-well plate, and 50 μL of indicator bacteria solution suspended in MH of 2 to 7×10⁵ CFU/mL was added to each well. Shaking culture was performed for at least 18 h, and an OD value was read at 600 nm with a microplate reader to obtain a minimum inhibitory concentration of the octapeptide. Five gram-negative bacteria strains were selected, which are Escherichia coli ATCC 25922, Salmonella gallinarum CVCC 534, Shigella dysenteriae CGMCC 1.1869, Pasteurella multocida CVCC 442 and Pseudomonas aeruginosa ATCC 27853 respectively, and five gram-positive bacteria strains were selected, which are Enterococcus faecalis ATCC 29212, Bacillus cereus CVCC 4101, Listeria monocytogenes CVCC 3746, Streptococcus dysgalactiae ATCC 35666 and Staphylococcus aureus ATCC 43300 respectively. Vancomycin and Colistin were used as positive controls respectively. Experimental results are shown in Table 7 below.

TABLE 7
MIC values of octapeptide inhibiting 10 indicator bacteria strains

| | | MIC values (μg/mL) | | |
|---|---|---|---|---|
| | Bacterial strain | Octapeptide | Vancomycin | Colistin |
| G− | Escherichia coli ATCC 25922 | 8 | 256 | 2 |
| | Salmonella gallinarum CVCC | 2 | 64 | 1 |
| | 534 | | | |
| | Shigella dysenteriae CGMCC | 4 | 128 | 1 |
| | 1.1869 | | | |
| | Pasteurella multocida CVCC | 4 | 128 | 1 |
| | 442 | | | |
| | Pseudomonas aeruginosa | 8 | >256 | 2 |
| | ATCC 27853 | | | |
| G+ | Enterococcus faecalis ATCC | 64 | 1 | >256 |
| | 29212 | | | |
| | Bacillus cereus CVCC 4101 | 16 | 1 | 256 |
| | Listeria monocytogenes | 8 | 1 | 64 |
| | CVCC 3746 | | | |
| | Streptococcus dysgalactiae | 8 | 2 | 64 |
| | ATCC 35666 | | | |
| | Staphylococcus aureus ATCC | 16 | 1 | >256 |
| | 43300 | | | |

Experimental results of antibacterial activity show that the octapeptide exhibits antibacterial activity against all of indicator bacteria. Activity against gram negative bacteria shows a MIC value range of 4 to 8 μg/mL. Activity against gram-positive bacteria is weak, which shows a MIC value range of 8 to 64 μg/mL. The activity of the octapeptide against gram-negative enterobacteria is generally better than that against gram-positive bacteria. A MIC value of the octapeptide to Salmonella gallinarum CVCC 534 strains was 2 μg/mL, which was very close to that against the positive control Colistin. To sum up, the octapeptide has good antibacterial activity against bacteria, and has research and development value as a drug lead compound and antibacterial peptide.

### Example 7 Stability Evaluation of Immunomodulatory Octapeptide in Pepsin Solution

Stability of anti-Enterobacter immunomodulatory octapeptide in the pepsin solution was determined by high-performance liquid chromatography and an inhibition zone method. According to Veterinary Pharmacopoeia of the People's Republic of China (edition in 2020), 16.4 mL of dilute hydrochloric acid was taken, about 800 mL of water was added and shaken with 10 g of pepsin, then transferred to a 1000 mL volumetric flask and diluted to a final volume of 1000 mL with ultrapure water to obtain artificial gastric juice. The immunomodulatory octapeptide (with a sample source: Example 3) was dissolved and diluted with the artificial gastric juice to a concentration of 200 μg/mL, placed in a water bath pot at 37° C., and samples was taken at 0, 0.5, 1, 2, 4 and 6 h respectively. After protease inactivating in water bath at 85° C. for 5 min, the sample was centrifuged at 9500 rpm and 4° C. for 15 min, and chromatographic peak areas of the octapeptide at different time points was measured to calculate a retention rate.

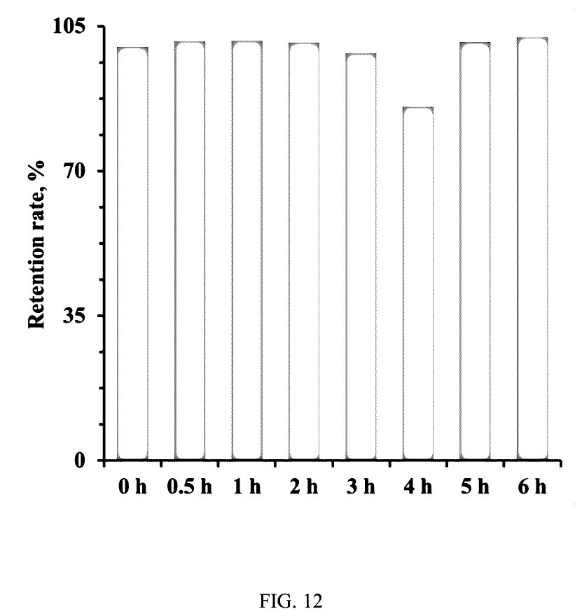

As can be seen from FIG. 12, the octapeptide can exist stably in the pepsin solution for 0 to 6 h, with a degradation retention rate remaining above 98.5%, showing excellent stability in the pepsin solution.

Polypeptide products have advantages of low toxic and side effects, high specificity, high drug activity and no ease of accumulating in bodies, which has attracted wide attention. However, polypeptides are highly sensitive to various hydrolases, including endoluminal enzymes from gastrointestinal and pancreatic secretions, bacterial enzymes and mucosal enzymes in colon, etc., and most of polypeptides degrade rapidly in simulated gastric juice. Therefore, oral administration of polypeptide is a great challenge. In the disclosure, the high-purity immunomodulatory octapeptide produced by Brevibacterium laterosporus and prepared by salt precipitation and high-performance liquid chromatography shows strong anti-pepsin degradation performance, which lays a foundation for its development as an oral drug and feed additive.

### Example 8 Hemolysis Evaluation of Immunomodulatory Octapeptide

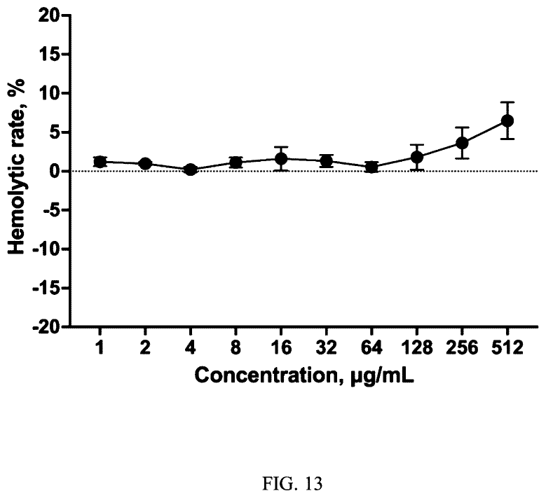

Pig whole blood was collected by vein using a blood collection tube with heparin sodium, and red blood cells were obtained after centrifugation. Blood cells were washed with 10 mM PBS (of pH 7.3) until supernatant had no obvious red color. The blood cells were prepared into a 4% suspension with PBS. 100 μL of 4% blood cells and 100 μL of double-diluted octapeptide solution with a concentration ranging from 0 to 512 μg/mL were added into wells of the 96-well plate, incubated in a thermostat at 37° C. for 1 h, centrifuged at 1500 rpm for 5 min, and the supernatant was transferred to a new 96-well plate, and an OD value was measured at 540 nm with a microplate reader. 10 mM PBS and 0.1% Triton X-100 were respectively used as negative and positive controls. Hemolysis of the octapeptide was calculated and obtained. Its calculation formula is as follows:

$$Hemolytic rate = \frac{{A}_{octapeptide} - {A}_{PBS}}{{A}_{0.1 \% Triton X - 100} - {A}_{PBS}} \times 100$$

in which A_{octapeptide} is an OD value of the octapeptide with different concentrations (with a sample source: Example 3) added into blood cell wells; APBS is an OD value of PBS added into the blood cell wells; and A0.1% Triton X-100 is an OD value of 0.1% Triton X-100 added into the blood cell wells. Results are shown in FIG. 13. It is known that hemolytic rates of the octapeptide in a concentration range of 1 to 512 μg/mL range from 0.201% to 6.477%, with changes within 7%. The results show that the octapeptide has no obvious hemolytic activity.

Immunomodulatory peptides or antibacterial peptides have potential to become new antibacterial drugs and feed additives instead of traditional antibiotics because of their simple structures, strong killing ability to bacteria and weak phenomenon of inducing bacteria to produce drug resistance. However, the immunomodulatory peptides also have some side effects such as hemolysis and instability. The hemolysis is an important index to evaluate cytotoxicity of the immunomodulatory peptides. The hemolytic evaluation shows that the immunomodulatory octapeptide did not show obvious hemolytic activity, which indicates that it has the potential to be further developed into new antibacterial drugs and feed additives.

To sum up, the immunomodulatory octapeptide produced in the disclosure has advantages of good antibacterial activity, stability, low toxicity and the like, and has broad application prospects in development of novel antibacterial drugs and feed additives.

The above only shows examples of the disclosure, which is not limited to the field involved in these embodiments. Common knowledge such as specific structures and features that are well-known in the schemes are not be described in detail herein. Ordinary skilled in the art know all common technical knowledge of the technical field to which the disclosure belongs before the application date or priority date, can know all existing technologies in this art, and have ability to apply conventional experimental means before that date. With inspiration given by this disclosure, the ordinary skilled in the art can improve and implement this scheme in combination with their own abilities. Some typical well-known structures or methods should not be obstacles for the ordinary skilled in the art to implement this disclosure. It should be noted that for those skilled in the art, several modifications and improvements can be made without departing from the structure of the disclosure, which should also be regarded as the protection scope of the disclosure and will not affect implementation effect and practicability of the disclosure. The protection scope claimed by this disclosure shall be subject to contents of the claims, and specific implementations in the specification can be used to explain contents of the claims.

1. An octapeptide, with a structural formula (I) as follows:

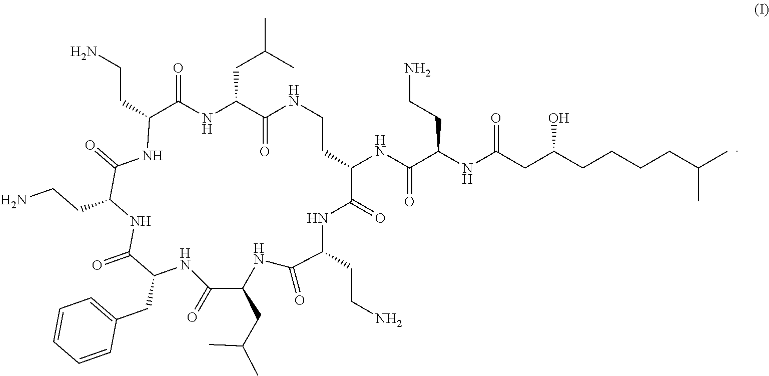 [SMILES: CC(C)CCCC[C@@H](O)CC(=O)N[C@H](CCN)C(=O)N[C@H]1CCNC(=O)[C@@H](CC(C)C)NC(=O)[C@@H](CCN)NC(=O)[C@@H](CCN)NC(=O)[C@@H](Cc2ccccc2)NC(=O)[C@H](CC(C)C)NC(=O)[C@@H](CCN)NC1=O]

2. Application of the octapeptide according to claim 1 in preparing antibiotics, feeds, or feed additives.

3. An antibiotic, feed, or feed additive, comprising the octapeptide according to claim 1.

4. The octapeptide according to claim 1, wherein the octapeptide is prepared by fermentation of Brevibacterium laterosporus, the brevibacterium laterosporus being preserved in China Center for Type Culture Collection on Apr. 25, 2024, with a preservation number of CCTCC NO: M2024715.

5. A preparation method of the octapeptide according to claim 1, comprising:
(1) fermenting brevibacterium laterosporus to prepare fermentation liquid, the brevibacterium laterosporus being preserved in China Center for Type Culture Collection on Apr. 25, 2024, with a preservation number of CCTCC NO: M2024715;
(2) preparing crude fermentation extract by salt ion precipitation;
(3) separating and purifying the crude fermentation extract by high-performance liquid chromatography, removing acetonitrile by rotary evaporation, and freeze-drying.

6. The preparation method of the octapeptide according to claim 5, wherein reversed-phase high-performance liquid chromatography is adopted in (3), and a separation and purification step is performed with a preparative liquid chromatographic column, Agilent Zorbax 300SB-C18 PrepHT; a flow of water containing 0.1% of trifluoroacetic acid (A) and acetonitrile containing 0.1% of trifluoroacetic acid (B); and
an elution mode being shown in Table 1:
TABLE 1
Elution gradient for octapeptide analysis

| Time | | |
|---|---|---|
| (min) | A, % | B, % |
| 0 | 70 | 30 |
| 57 | 63.4 | 36.6 |
| 58 | 0 | 100 |
| 64 | 0 | 100 |
| 65 | 70 | 30 |
| 67 | 70 | 30 |

with a flow rate of 5 mL/min, column temperature of 30° C., and a detection wavelength of 220 nm.

7. The preparation method of the octapeptide according to claim 5, wherein the salt ion precipitation comprises adding ammonium sulfate with a final concentration of 20 g/mL into centrifuged supernatant of the fermentation liquid, centrifuging and drying to obtain crude octapeptide extract.

8. A testing method of the octapeptide according to claim 1, comprising:
treatment of test samples: shaking fermentation liquid of the octapeptide evenly, taking out 1 mL of the fermentation liquid using a pipette and placing it in a 2 mL centrifuge tube, adding 20 μL of 5M sulfuric acid for vortexing for 1 min, centrifuging at 12000 rpm for 5 min, and passing supernatant through a 0.22 μm membrane for filtering for reversed-phase liquid analysis; and
analysis and measurement of analytical high-performance liquid chromatography, which is performed with an analytical liquid chromatography column, Sepax Bio-C18 4.6×250 mm 5 μm; and a flow of water containing 0.1% of trifluoroacetic acid and acetonitrile containing 0.1% of trifluoroacetic acid;
an elution mode being shown in Table 2:
TABLE 2
Elution gradient for octapeptide analysis

| Time | | |
|---|---|---|
| (min) | A, % | B, % |
| 0 | 80 | 20 |
| 4 | 80 | 20 |
| 10 | 50 | 50 |
| 13 | 40 | 60 |
| 15 | 0 | 100 |
| 19 | 0 | 100 |
| 22 | 80 | 20 |
| 26 | 80 | 20 |

with a flow rate of 1 mL/min, column temperature of 30° C., and a detection wavelength of 220 nm.


SEQUENCE LISTING (2 sequences)

SEQ ID NO: 1 - DNA, 1577 bp, synthetic construct
  source 1..1577: mol_type = other DNA
ataaaacgaaagtgaggcggttctatatgataatttgtaatgacgattgtacgactatatgcgacttctgcgtacacttccaagatagtcacatgctttcggaagagtgcgacggcatttgctcccgaactggcggtgagacgtacgcaacgagtacttgcgacagtttccattgtattcgcgtatagatcagaaccgccggggctcccctgacgtacttacggaggcgtcgtaacccaacagacggtgaaccaccgcaaattgccgcacatgtaattccggaaagcctaacgcgtaatgtccgcctttccattcgagcttgtacgggatcgcagcggaatctagcaccgggagaatcagcgagaggacttcgtgagaaacggtgtaagaaaacgaaatattcaatgtagtccacactcctttactgcgcattatagcgggtagggttgggacaaaaaaaagaaaacaaacgccaagggtggaagacacacgcttagcaatgagaggggtgaacgaagtgataaaaatacttgaattattcggaggaattggagcaccgaggaaggcgcttgttaacttaggaatagatcataaagcaattgattatgtggaatggcaagtgaacagagtaaaggcatataacgctctttatgaccatctacacaaaccacaagatgttaaaggatggaacttaaagcctgacattctagttcatggatcaccttgtcaagataacagtagggcaaataaaaaacgcaaaggtactgctaaagattcacgttcgcaattgatgcttgaaacgttgcggataatcaaagaaatgggtgaatggcgaccgaaagttgttatttgggagaacgtgaaaggcgtgcttgatcgtaatgtagtgccagtttttaattattatcttcaagaaatgaaaatgttaggttatacaaacgcctttgatgtattggatgctagaaaattcggtatcccccatgcgagagaaagagtgttttgcatctcattattaggaaatgaaacatttgattttagtaagcttcggtataaaccaatgaagaacattaaagagtttttagagtatggaccagatgatgagattccatcacaatacttaattaatattccgtcgatgttaagtaaaattaaagaatttaatccaagggcaagtgggagttataagcgacagttagatgttattaatgattattgttatacgattaccgaaagacaagatagatgcccaaacgccggaattattcgactaaacaaaccgcaatatcgttatttaacagagcgtgagtgttggcgattattaggttttgatgatgaagattatgagcttatgttaaaagagtttccaagtaagaaagggaagcgtaatgcaactctttatgctctagctggaaatagcattgtagtcgatgtgttagaagcaatttttgaggtgctactgacagaaaaccacgggcaaatagaaatcaatactaacagatccggtcagctggaattggtttgttaatctattcaacggccagcagatcatttggaacaaaaataggaggttagtagac

SEQ ID NO: 2 - DNA, 1500 bp, synthetic construct
  source 1..1500: mol_type = other DNA
agagtttgatcctggctcaggacgaacgctggcggcgtgcctaatacatgcaagtcgagcgagggtcttcggaccctagcggcggacgggtgagtaacacgtaggcaacctgcctgtaagactgggataacatagggaaacttatgctaataccggatagagttttgcttcgcatgaagcgaaacggaaagatggcgcaagctatcacttgcagatgggcctgcggcgcattagctagttggtgaggtaaaggctcaccaaggcgacgatgcgtagccgacctgagagggtgaccggccacactgggactgagacacggcccagactcctacgggaggcagcagtagggaattttccacaatggacgaaagtctgatggagcaacgccgcgtgaacgatgaaggctttcgggtcgtaaagttctgttgttagggaagaaacagtgctatttaaataaggtagcaccttgacggtacctaacgagaaagccacggctaactacgtgccagcagccgcggtaatacgtaggtggcaagcgttgtccggaattattgggcgtaaagcgcgcgcaggtggctatgtaagtctgatgttaaagcccggggctcaacctcggttcgcattggaaactgcgtagcttgagtgcaggagaggaaagtggtattccacgtgtagcggtgaaatgcgtagagatgtggaggaacaccagtggcgaaggcgactttctggcctgtaactgacactgaggcgcgaaagcgtggggagcaaacaggattagataccctggtagtccacgccgtaaacgatgagtgctaggtgttaggggtttcaatacccttagtgccgcagctaacgcaataagcactccgcctggggagtacgctcgcaagagtgaaactcaaaggaattgacgggggcccgcacaagcggtggagcatgtggtttaattcgaagcaacgcgaagaaccttaccaggtcttgacatcccactgaccgctctagagatagagcttcccttcggggcagtggtgacaggtggtgcatggttgtcgtcagctcgtgtcgtgagatgttgggttaagtcccgcaacgagcgcaacccttatctttagttgccagcattcagttgggcactctagagagactgccgtcgacaagacggaggaaggcggggatgacgtcaaatcatcatgccccttatgacctgggctacacacgtgctacaatggttggtacaacgggatgctacttcgcgagaagatgctaatctcttaaaaccaatctcagttcggattgtaggctgcaactcgcctacatgaagtcggaatcgctagtaatcgcggatcagcatgccgcggtgaatacgttcccgggccttgtacacaccgcccgtcacaccacgggagtttgcaacacccgaagtcggtgaggtaaccgcaaggagccagccgccgaaggtggggtagataactggggtgaagtcgtaacaaggtagccgta